(19) United States
(12) Patent Application Publication
(10) Pub. No.: US 2009/0124918 A1
(43) Pub. Date: May 14, 2009

(54) Apparatus For Spectroscopically Analyzing A Gas

(75) Inventors: Martin Stockmann, Berlin (DE); Karsten Heyne, Grossbeeren (DE); Bjoern Riecke, Hamburg (DE)

Correspondence Address: Workman Nydegger;1000 Eagle Gate Tower, 60 East South Temple, Salt Lake City, UT, 84111, US

(73) Assignees: Freie Universitaetsmedizin Berlin,, Berlin (DE); Charite Universitaetsmedizin Berlin,, Berlin (DE)

(21) Appl. No.: 12/293,265

(22) PCT Filed: Mar. 16, 2007
(86) PCT No.: PCT/EP2007/002525

§ 371 Date: Nov. 12, 2008

(30) Foreign Application Priority Data
Mar. 17, 2006 (DE) 10 2006 012 740.4
Apr. 13, 2006 (DE) 10 2006 018 862.4

(51) Int. Cl. A61B 5/08; G01N 21/63

(52) U.S. Cl. 600/532; 356/437

(57) ABSTRACT

The invention relates to an apparatus for spectroscopically analysing a gas, said apparatus having at least one radiation source (1), at least one detection apparatus (12; 20), at least one sample chamber (13) and a system of optical elements (4; 5; 6; 7; 9; 10; 11; 18; 19) which is intended and set up to direct at least part (3b) of the radiation (3) emitted by the radiation source (1) through the sample chamber (13) onto the detection apparatus (20), wherein the sample chamber (13) is used to hold a gaseous sample which contains the gas to be analysed, and wherein the apparatus is configured in such a manner that the sample can continuously flow through the sample chamber (13), and means (16) are provided for the purpose of determining the pressure and/or the volume and/or the concentration of the sample in the sample chamber (13). The invention also relates to a corresponding method for spectroscopically analysing a gas.

The invention relates to an apparatus for the spectroscopic analysis of a gas according to the preamble of claim 1, to a method for the spectroscopic analysis of a gas according to the preamble of claim 18 and to the use of an apparatus according to the invention according to the preamble of claim 28.

The analysis of a gas has very many possible applications, particularly in medicine. The concentration of ¹³CO₂ is often studied, for example, in the respiratory air of patients who have previously been administered with ¹³C-labeled substances which are converted by the body and lead to the production of ¹³CO₂ (¹³C breath tests). Such studies are suitable for example for the diagnosis of Helicobacter pylori, for measurements of the gastric emptying time or for liver function tests.

In the prior art the concentration of ¹³CO₂ is determined by mass spectrometry, Fourier transform infrared spectrometry or by direct inorganic chemical analysis. The use of said techniques generally requires great outlay on expensive instruments or equipment, which cannot be used directly on the patient. For this reason, nondispersive isotope-selective infrared spectroscopy (NDIRS) (for example Fischer Analysen Instrumente, Leipzig) and a method based on infrared emission and absorption (LARA) are also used in the prior art. Both methods, however, measure only relative ¹³CO₂ concentration changes and do not allow an absolute ¹³CO₂ concentration measurement. In the latter two methods, an estimated total CO₂ production rate of a patient being examined is used as a basis for calculating the relative ¹³CO₂ concentration changes, without it being possible to determine the actual total CO₂ production exactly.

The NDIRS method is sensitive enough to measure for example the relative ¹³CO₂ concentration changes in the respiratory air of patients, but in the event of different carrier gas mixtures (for example O₂) it gives varying results which are therefore difficult to evaluate and it therefore allows only very limited resolution of the ¹³C metabolism owing to its slow measurement method. The measurement accuracy of NDIRS is likewise limited in this case, and in particular is insufficient especially for directly quantitative measurements such as determination of the quantitative liver-function capacity when other measurement effects such as varying carrier gases are added (Perri, F., R. M. Zagari, et al. (2003). “An inter- and intra-laboratory comparison of ¹³CO₂ breath analysis. Aliment. Pharmacol. Ther. 17(10): 1291-7). Furthermore, NDIRS instruments cannot be used in a mobile fashion.

Furthermore, effectively only the ¹³CO₂/¹²CO₂ ratio is determined owing to method limitations. The absolute amount of ¹³CO₂ expired per unit time can be calculated from this with the aid of the patient's measured CO₂ production rate per minute. In a single individual, however, the CO₂ production rate can be measured directly only with great difficulty. For calculation in the prior art, an estimated standard value of the CO₂ production rate is therefore used which is respectively adapted to the body surface area of the individual (Schoeller, D. A., J. F. Schneider, et al. (1977). “Clinical diagnosis with the stable isotope ¹³C in CO2 breath tests: methodology and fundamental considerations.” J. Lab. Clin. Med. 90(3): 412-21; Schoeller, D. A., P. D. Klein, et al. (1981). “Fecal ¹³C analysis for the detection and quantitation of intestinal malabsorption. Limits of detection and application to disorders of intestinal cholylglycine metabolism.” J. Lab. Clin. Med. 97(3): 440-8). This method leads to considerable inaccuracy in many clinical studies, in which the CO₂ production rate of the individual is varied relative to the normal state.

US 2004/0211905 A1 describes a breath analyzer in which parts of exhaled respiratory air are introduced through a gas exchange system into a spectrometer for analysis. Only the relative ratio of two isotopes of a gas relative to one another can be determined in the analyzer, but not the absolute concentration of one isotope alone. Preferably not all of the exhaled air, rather only parts of it, are introduced into the spectrometer by using the gas exchange system.

U.S. Pat. No. 6,186,958 describes a breath analyzer which is designed for the online analysis of continuously exhaled respiratory air. By using a plurality of gas discharge lamps, which are respectively filled with only one isotope of a gas to be analyzed, this analyzer can distinguish between individual isotopes of the gas. Even by means of this analyzer, however, it is only possible to determine the relative ratio of the individual isotopes of the gas with respect to one another. This is due in particular to the fact that the concentration of the respiratory air to be analyzed in a sample chamber of the analyzer cannot be determined.

It was an object of the present invention to provide an apparatus which is suitable for determining the absolute concentration of a gas in a gas mixture; to develop a method by means of which such determination is carried out as well as to provide a suitable use for an apparatus according to the invention.

This object is achieved by an apparatus having the features of claim 1, a method having the features of claim 18 and the use of an apparatus according to the invention having the features of claim 28.

Such an apparatus for the spectroscopic analysis of a gas comprises at least one radiation source, at least one detection apparatus, at least one sample chamber and a system of optical elements, which is intended and adapted to direct at least a part of the radiation emitted by the radiation source through the sample chamber onto the detection apparatus, the sample chamber being used to receive a gaseous sample which contains the gas to be analyzed. This apparatus is distinguished in that it is configured so that the sample can flow continuously through the sample chamber, and so that means are provided for determining the pressure and/or the volume and/or the concentration of the sample in the sample chamber.

Such means may for example be a pressure meter or a volume meter, optionally in conjunction with a temperature sensor.

The system of optical elements consists of lenses, mirrors, filters and beam splitters and comparable elements, their number and sequence in the beam path of the apparatus being freely selectable so long as the desired guiding effect is achieved. In general only as many optical elements are used as are required for best possible performance of the apparatus.

In a preferred configuration of the invention, the apparatus for the spectroscopic analysis of a gas is configured so that essentially only absorption of a single isotope of the gas is excited by the emitted radiation and/or recorded by the detection apparatus.

In order to achieve this, the emitted radiation preferably passes through a filter which only transmits radiation in the desired wavelength range. A narrowband detection apparatus is furthermore preferably used, which is particularly sensitive in the wavelength range to be analyzed and the detection power of which is essentially unaffected by radiation possibly incident with a different wavelength. A radiation source which only emits radiation in a narrow wavelength range may also preferably be used, so that essentially no absorption other than the desired absorption is excited. The aforementioned functional elements may be used individually or in any desired combination in an apparatus according to the invention, in order to achieve the essentially isotope-selective excitation.

In order to allow a high information density of the gas analyses carried out by means of the apparatus, the apparatus is preferably configured so that the spectroscopic analysis is carried out with time resolution. To this end a radiation source which emits pulsed light is preferably used, or a chopper which can convert continuous radiation by interrupting the light beam into radiation with a defined repetition rate is positioned in the beam path.

The time resolution is preferably better than 1 second and particularly preferably between 0.2 and 0.4 second (for example 0.3 second or better). More than 3 measurements can thus be carried out per second with a preferred alternative embodiment of the invention, which results in fine graduation of a time profile of the analysis being carried out.

Since in particular molecular vibrations are intended to be studied, the radiation source preferably emits light with a wavelength in the infrared range, medium infrared being particularly preferred. Medium infrared has a wavelength of about 2.5 to 50 micrometer (corresponding to 4000 to 200 cm⁻¹).

So that pulsed radiation with a high energy density and brilliance is directly emitted by the radiation source, a quantum cascade laser is preferably used.

For an application of the apparatus according to the invention to study ¹³CO₂ absorptions, which represents a preferred use of the invention, a quantum cascade laser which emits light in a wavenumber range of about 2280 to 2230 cm⁻¹ is preferably suitable. The P branch of ¹³CO₂ in the gas phase absorbs in this wavenumber range, while essentially no other interfering absorptions for example by ¹²CO₂, H₂O or O₂ can be observed.

For the sensitive and specific absorption of the ¹³CO₂ bands preferably being studied, it is preferable to use a photovoltaic mercury cadmium telluride detector (MCT detector) which does not require cooling with liquid nitrogen. A detection maximum of the detector around 2270 cm⁻¹ is advantageous.

Since ¹³CO₂ has only a small absorption, albeit without interference, in the spectral range preferably being studied, the sample chamber preferably contains a multiplicity of mirrors which reflect the light beam input into the sample chamber repeatedly to and from inside the sample chamber. In this way, the beam path traveled by the light beam is increased by a multiple and the amount of gas studied is virtually increased. This method may also be applied to other substances which only have a low extinction coefficient in the range respectively studied.

The mirrors are preferably arranged so that the beam path to be traveled by the light beam inside the sample chamber is longer than 1.5 m and up to 2.5 m or more. The sample chamber per se, on the other hand, is only a few centimeters or decimeters large.

The sample to be studied is preferably respiratory air, which contains the gas to be analyzed. The respiratory air is preferably exhaled directly into the apparatus by an individual, so that the respiratory air is exhaled air.

In a preferred configuration of the invention, the gas to be analyzed is ¹³CO₂.

The exhaled respiratory air or another sample is preferably transferred by means of a tube, which in a preferred configuration is heated in order to prevent water from accumulating in the tube, and in order to guarantee that the gas temperature remains constant. To ensure reliable functional integrity of the apparatus, it is preferably configured so that only specially developed tubes can be connected to the apparatus. Optionally, a first adapter is to be used for the connection. If respiratory air is to be analyzed as the sample, then it is expedient to provide the tube with a second adapter in the form of a mouthpiece in order to allow respiratory air to be blown easily into the tube.

So that the sample flowing into the sample chamber can also leave the sample chamber again, it is preferably provided with a gas outlet means which makes it possible for the sample to flow out of the sample chamber. The gas outlet means is configured so that it allows the sample or another substance to flow out of the sample chamber, but does not allow the sample or substance to flow into the sample chamber. The gas outlet means may for example be configured so that when there is a particular pressure in the sample chamber, it opens and allows the sample to flow out of the sample chamber. This pressure may be only a little higher than the normal ambient air pressure.

A method for the spectroscopic analysis of a gas comprises the following steps: introducing a sample, which contains the gas to be analyzed, into a sample chamber by the sample flowing into the sample chamber, the sample chamber allowing subsequent flow of the sample out of the sample chamber, directing at least a part of radiation emitted by a radiation source through the sample chamber onto a detection apparatus by means of a system of optical elements for analysis of the gas and detecting absorption of the radiation by the gas to be analyzed by means of the detection apparatus. Such a method is distinguished in that a variation of the pressure and/or volume and/or concentration of the sample in the sample chamber during the analysis is determined by suitable means.

Preferably, essentially only absorption of a single isotope of the gas is excited by the emitted radiation and detected by the detection apparatus. In conjunction with determining the pressure, volume or concentration change of the sample in the sample chamber during the analysis, it is thus possible to determine the absolute concentration of an isotope of the gas.

In a preferred application of the method, the spectroscopic analysis is carried out with time resolution in order to obtain analytical measurement values as a function of time. In this way, for example, it is possible to determine concentration changes of the gas to be analyzed over the time duration of the analysis.

The time resolution is preferably better than 1 second and particularly preferably between 0.2 and 0.4 second (for example 0.3 second or better). With such a time resolution, even rapid metabolic processes can still be studied accurately without entailing the risk of a significant information loss due to averaging or non-detection of various states owing to excessively long measurement intervals.

Absorption of the gas to be analyzed is preferably detected in the medium infrared range, detection in the wavenumber range of from 2230 to 2280 cm⁻¹ being particularly preferred.

In a preferred configuration of the invention, the sample to be analyzed is exhaled respiratory air, the gas to be analyzed preferably being ¹³CO₂.

The respiratory air is preferably introduced into the sample chamber using a tube, which is heated in order to avoid condensation of gaseous constituents of the sample on the inner wall of the tube or accumulation of liquid constituents of the sample there, and to ensure thermal regulation of the sample.

In a preferred configuration of the invention, the sample flows out of the sample chamber through an outlet means, which prevents substances from being able to enter the sample chamber. The outlet means thus allows exclusive sample transport out of the sample chamber.

The apparatus according to the invention is suitable for the determination of a biological parameter of an individual, a spectroscopic analysis of a gaseous sample originating from an individual being carried out for this determination. In particular exhaled respiratory air may be envisaged as a gaseous sample. The sample is analyzed outside the individual's body.

The biological parameter is preferably the function of an organ of the individual, function and capacity determinations of the liver and the pancreas being particularly preferred.

In a variant of the invention, the apparatus may also be used to determine the concentration of an enzyme, for example lactase, by means of analyzing the individual's respiratory air and thus being able to draw conclusions about enzyme deficiency states of the individual.

In another variant of the invention, the apparatus may also be used to determine the concentration of a microbial species, for example a particular bacterium, a virus or a fungus in an organ or a tissue of the individual. This may preferably involve determining the Helicobacter pylori concentration in the individual's stomach.

Other advantages and details of the invention will be explained in more detail with the aid of drawings, in which:

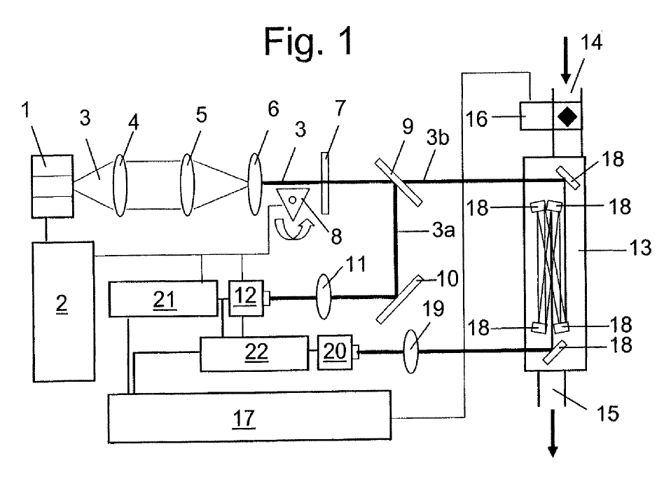

FIG. 1 shows a schematic representation of the structure of an apparatus according to the invention for the spectroscopic analysis of the gas,

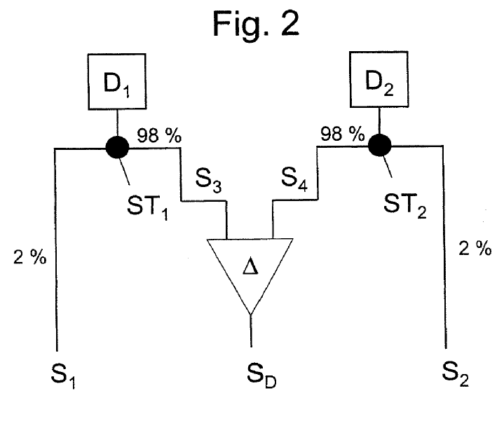

FIG. 2 shows a diagram for the calculation of a difference signal, based on signals which are detected by an apparatus according to FIG. 1, and

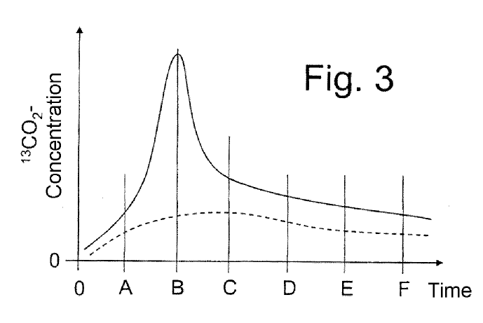

FIG. 3 shows a schematic representation of possible profiles of the ¹³CO₂ concentration in exhaled respiratory air.

FIG. 1 shows a schematic representation (not true to scale) of an infrared spectrometer as an exemplary embodiment of an apparatus according to the invention for the spectroscopic analysis of a gas.

The infrared spectrometer comprises a radiation source 1 in the form of a laser or a globar and a driver 2 for the radiation source 1, which is electronically connected to the radiation source 1. Radiation is emitted by the radiation source 1 in the form of a light beam 3, which has a wavelength in the medium infrared. After it leaves the radiation source 1, the light beam 3 initially strikes a cylindrical lens 4 which ensures parallel propagation of the light beam 3. After a variable distance, it strikes a first lens 5 which is arranged on the same optical axis as the cylindrical lens 4 and focuses the light beam 3 onto a second lens 6, which is likewise arranged on the same optical axis as the cylindrical lens 4 and the first lens 5. The second lens 6 ensures highly collimated, essentially parallel propagation of the light beam 3.

In the further course of its propagation, the light beam strikes a filter 7 which transmits only that part of the light beam 3 which is intended to be used for the detection of a sample. In this exemplary embodiment, the filter 7 is a narrowband infrared filter which only transmits light with a wavelength corresponding to a wavenumber of about 2260±20 cm⁻¹.

A chopper 8, which is employed in particular whenever a globar is used as the radiation source 1, is arranged between the second lens 6 and the filter 7. While a laser can directly emit pulsed radiation, the radiation which is emitted by a globar is continuous unpulsed radiation. Owing to the chopper 8, which is electronically connected to the driver 2 of the radiation source 1, the radiation emitted by a globar can also be pulsed.

The radiation emitted by a preferably used quantum cascade laser has a repetition rate of 10 kHz. If a globar is used instead of the laser, then a repetition rate of about 10 kHz is set up by means of the chopper 8.

After the light beam 3 has passed through the filter 7, it strikes a beam splitter 9 which splits the light beam 3 into a first sub-beam 3a and a second sub-beam 3b. The first sub-beam 3a is deviated through 90° by the beam splitter, while the second sub-beam beam 3b passes through the beam splitter in continuation of the original propagation direction of the light beam 3. The first sub-beam 3a is directed by means of a deviating mirror 10 and a third lens 11 onto a first detector 12, which detects the intensity of the first sub-beam 3a.

The second sub-beam 3b is directed into a sample chamber 13. The sample chamber 13 is filled with a gaseous sample, which is supplied to the sample chamber 13 through a gas inlet 14 in the direction of the arrow and can leave the sample chamber 13 through a gas outlet 15 in the direction of the arrow. The gas outlet 15 is configured so that no gas can enter the sample chamber 13 through the gas outlet. By means of a gas flow meter 16, the volume of gas supplied to the sample chamber 13 through the gas inlet 14 is measured so that the quantity of gas contained in the sample chamber 13 is always accurately known. The gas flow meter 16 is electronically connected to a computer 17 and can transfer the data which it acquires to the computer 17.

The sample chamber 13 contains a system of a plurality of mirrors 18, which direct the second sub-beam 3b to and from inside the sample chamber 13 so that the beam path of the second sub-beam 3b in the sample chamber is lengthened relative to the actual length dimension of the sample chamber 13. Lastly, one of the mirrors 18 directs the second sub-beam 3b back out of the sample chamber. After passing through a fourth lens 19, the second sub-beam 3b strikes a second detector 20 by which the intensity of the second sub-beam 3b is detected.

Because the intensity of the first sub-beam 3a, which does not experience any attenuation by an absorbing substance, is always measured in parallel with the intensity of the second sub-beam 3b which is attenuated by the absorption of the sample 13 in the sample chamber, it is possible to compensate for minor intensity differences of the radiation 3 emitted by the radiation source 1. Measurement errors, which could occur owing to such a minor intensity differences, can be avoided in this way.

The first detector 12 is electronically connected to a first lock-in amplifier 21 and to a second lock-in amplifier 22. The second detector 20 is connected to the second lock-in amplifier 22. The two lock-in amplifiers 21 and 22 are used to amplify the relatively weak intensity signals of the two sub-beams 3a and 3b as detected by the two detectors 12 and 20. Both lock-in amplifiers are part of an electronic component module of the infrared spectrometer, which also includes the driver 2 of the radiation source 1, the chopper 8, the gas flow meter 16, the first detector 12, the second detector 20 and the computer 17.

Inside the electronic component module, the chopper 8 is electronically connected directly to the drive 2 of the radiation source 1, the first detector 12, the first lock-in amplifier 21 and the second lock-in amplifier 22. Furthermore, the first lock-in amplifier 21 and the second lock-in amplifier 22 are connected directly to one another and to the computer 17. The respective electronic connections are used for data interchange and synchronization of the individual components with one another. The computer 17 is used to display and evaluate the acquired data.

By using pulsed light with a repetition rate of about 10 kHz, it is possible to detect lock-in-amplified signals with a time resolution of about 0.3 second. The advantages of such a time resolution will be explained in more detail in the description of FIG. 3.

As a filter 7 in order to determine the ¹³CO₂ content in a sample, a narrowband infrared filter is used which limits the infrared component light that can pass through the filter to those wavelengths in which ¹³CO₂ exhibits characteristic absorption bands. This is preferably the wavelength range which corresponds to wavenumbers of from 2280 to 2230 cm⁻¹. It is also possible to use a filter which only transmits light in a wavelength range that corresponds to wavenumbers of from 2282 to 2250 cm⁻¹.

The first detector 12 and the second detector 20 are both photovoltaic mercury cadmium telluride detectors (MCT detectors) with a peak response sensitivity of 1.6 A/W. In contrast to conventional MCT detectors, these MCT detectors do not have to be cooled with liquid nitrogen. Instead, the cooling is carried out by means of a Peltier element. An average power of about 0.3 mW distributed over 40 cm⁻¹ for a laser as the radiation source 1 gives a measurement signal of a few hundred μA. The noise of each of the two lock-in amplifiers 21 and 22 lies in the pA range, and therefore far away from the signal range. The signal can thus still be attenuated strongly—without entering the noise range.

Assuming ¹³CO₂ absorption with an absorption coefficient ε=30 m²/mol and a ¹³CO₂ concentration of about 1.4·10⁻⁴ mol/m³ in normal ambient air, an absorption of about 0.0042 per meter by the ¹³CO₂ may be estimated. The beam path in the sample chamber 13, which contains the gas, is therefore several meters (for example 1.5 to 2.5 m) in order to ensure sufficient absorption of the incident second sub-beam 3b by the ¹³CO₂.

Compared with the prior art, the following advantages and improvements are achieved by an apparatus according to the invention as described in FIG. 1:
It is possible to carry out measurements of the absolute concentration of a gas per time interval.
The concentration measurement takes place more rapidly, so that faster evaluation of the data is also possible.
The data reliability is greater owing to a lower susceptibility to fluctuations.
Concentration changes can be tracked in real-time.
The flow measurement technique allows continuous measurement of the gas samples.
The ¹³CO₂ concentration is measured independently of the ¹²CO₂ concentration.
The measurement results are independent of most carrier gases. Thus, carrier gases which are employed in anesthesia may also be used as carrier gases.
The apparatus can be used directly on a patient.
A compact design allows mobile use.
Precisely measuring the ¹³CO₂ concentration and obviating an estimate of the CO₂ production rate permit more accurate quantitative inferences (for example quantitative inferences about the liver-function capacity)

In conjunction and with reference to the infrared spectrometer represented in FIG. 1, FIG. 2 shows a diagram for the calculation of a difference signal S_{D} based on two individual signals D₁ and D₂, which are detected by the first detector 12 and the second detector 20. Numerical references refer to FIG. 1, while letters as references refer to FIG. 2.

Only about 1% of the infrared light shone into the sample chamber 13 as the second sub-beam 3b is absorbed at the absorption wavelengths of ¹³CO₂. In this signal, an absorption change of less than 1% is intended to be measured. This is done by measuring the signal D₁ of the first detector 12 and the signal D₂ of the second detector 20, with subsequent differencing Δ. Since the two detector signals D₁ and D₂ are much greater than their difference S_{D}, only a first sub-signal S₁ or S₂ which covers a few percent (preferably about 2%) of the signal D₁ or D₂, respectively, is used for the direct measurement. This splitting of the signals D₁ and D₂ into a first sub-signal S₁ and S₂ respectively, and a second sub-signal S₃ and S₄ respectively, is carried out by using two voltage dividers ST₁ and ST₂.

The difference signal S_{D} is measured using the sub-signals S₃ and S₄, which respectively cover the main components of the detector signals D₁ and D₂. The two signals S₁ and S_{D} are amplified by the first and second lock-in amplifiers 21 and 22 respectively (or alternatively in a one-shot measurement with integrated preamplifiers) and converted into digital signals by an analog-digital converter in the computer 17. The desired measurement signal of the absorption in the sample chamber A=−log(D₂/D₁) is determined by recording −log(11−S_{D}/S₁)=εcd−φ.

Here ε is the extinction coefficient of ¹³CO₂, c is the concentration and d is the beam path of the second sub-beam 3b in the sample chamber 13. The constant parameter φ contains structural parameters, for example the splitting ratio of the beam splitter 9 and the base ¹³CO₂ concentration in the infrared spectrometer. The measurement signal thus directly delivers the desired ¹³CO₂ concentration c of the sample for known (and constant) values ε, d and φ. For installation and maintenance, standardization of the infrared spectrometer may readily be carried out with known ¹³CO₂ concentrations. The absorption data are correlated with the gas flow meter 16, so that adaptation to the concentration differences of the sample in the sample chamber 13 can be carried out.

This manner of data acquisition makes it possible to utilize the high sensitivity of the first and second detectors 12 and 20, the two lock-in amplifiers 21 and 22 and the analog-digital converter. The overall equipment structure of the infrared spectrometer with the sample chamber 13 and said electronic elements is compact, transportable and insensitive to external effects. This further increases the range of use.

FIG. 3 schematically shows two profiles of the ¹³CO₂ concentration in exhaled respiratory air, plotted over a time frame of a few seconds. Such profiles can be determined by means of an apparatus according to the invention, as represented in FIG. 1.

While the ¹³CO₂ concentration in the respiratory air of an individual with a healthy liver, after application of a ¹³C-labelled substance metabolizable to ¹³CO₂ in the individual's liver, rises very rapidly after application of the substance and then returns to a low level (solid curve), the ¹³CO₂ concentration in the respiratory air of an individual with a diseased liver reaches only very low values after application of the substance, before subsequently approaching a level which is comparable with or lower than that of the individual with a healthy liver (dashed curve).

Depending on the nature and severity of the liver disease, it is possible to find various curved profiles which inter alia may be very similar to that of a healthy liver. Only by measurement with a high time resolution—preferably in the subsecond range, as is possible with an apparatus according to the invention—can the curves represented in FIG. 3 be determined accurately enough, as represented by specifying exemplary measurement instants A to F. If a comparable study were to be carried out with a device which can measure only at the measurement instants C and F, for example owing to an inferior time resolution, then the results integrated over the periods 0 to C and C to F would respectively be obtained.

This would mean that discrimination between individuals with healthy and diseased livers could only be carried out insufficiently. This would be the case in particular when, instead of the linear profile of the two curves beyond the measurement instant E as represented in FIG. 3, level differences still occur which could quite feasibly remain undiscovered by mutual cancellation in the event of an integrated measurement due to inferior time resolution.

The use of an apparatus according to the invention—for example as represented in FIG. 1—will be explained in more detail below with the aid of application examples.

### EXAMPLE 1

### Use as a Breath Analyzer for Liver-Function Determination

Although application of an apparatus according to the invention is not restricted to breath tests alone, but instead may generally be used for the analysis of any gas mixtures, use in breath analysis is suitable.

Thus, the liver function of an individual may be determined quantitatively with an apparatus according to FIG. 1. Such determination is of great importance in many fields of medicine. Chronic liver diseases are widespread in Europe, 8.9 million people being infected with hepatitis C alone. These patients with progressive disease are usually in constant medical care. In the therapy and management of patients with chronic liver diseases, significantly improved therapy can be carried out by quantification of the liver function. Estimating the liver function is crucial for making suitable therapy decisions.

Partial liver resection is a conventional method in modern surgery. It is carried out as a segment resection or hemihepatectomy along the anatomical boundaries. Extended interventions in the parenchymatous organ have been made possible by the development of a wide variety of operation techniques. The postoperative morbidity and mortality due to liver failure owing to deficient liver-function capacity in the event of predamaged or insufficient remaining liver tissue is however a significant problem. A large number of operative interventions must however be carried out in a predamaged liver tissue, usually a cirrhotically altered liver.

It is therefore of great importance that a patient's functional liver capacity can already be determined before partial liver resection, so that patients who no longer have a sufficient functional reserve of their liver tissue are not exposed to the operation risk which is too great for them, so that other therapy methods can be carried out. Estimating the liver function is of great importance in liver transplantation, since here the organ function must be estimated promptly and a rapid therapy decision must be made. Here, furthermore, in many clinical situations it is very difficult to estimate whether there is parenchymatous function impairment or whether other causes are responsible for the patients' clinical symptoms. To summarize, there is therefore a great need to provide a truly quantitative liver function test for wide application in medicine. By a breath test with for example ¹³C-labeled methacetin, this is possible when the quantity of ¹³CO₂ exhaled per time interval in the exhaled air can be measured absolutely and precisely. Previous tests could only achieve semiquantitative results owing to unfavorable administration (orally) and insufficient measurement methodology (Matsumoto, K., M. Suchiro, et al. (1987). “[¹³C] methacetin breath test for evaluation of liver damage.” Dig. Dis. Sci. 32(4): 344-8; Klatt, S., C. Taut, et al. (1997). “Evaluation of the ¹³C-methacetin breath test for quantitative liver function testing.” Z. Gastroenterol. 35(8): 609-14). With corresponding application (intravenously), new calculation and accurate absolute concentration measurement by means of an apparatus according to the invention, widespread progress in this field is possible.

### EXAMPLE 2

### Use as a Breath Analyzer for Determining Other Parameters

A further application of an apparatus according to the invention is to measure the gastric emptying time. The gastric emptying time is affected by many gastrointestinal diseases (gastroparesis). This may be the case for example with diabetic gastropathy, dyspepsia or other diseases. In order to measure the gastric emptying time, a ¹³C-labeled test substance (for example octanoic acid) is administered by a test meal and the exhalation of ¹³CO₂ is also measured. Here, continuous measurement by means of an apparatus according to the invention likewise offers much better accuracy in the analysis of the data.

¹³CO₂ measurements have further applications in the diagnosis of pancreatic diseases, in the diagnosis of Helicobacter pylori and in the diagnosis of enzyme deficiency states (lactase deficiency etc.) (Swart, G. R. and J. W. van den Berg (1998). “¹³C breath test in gastroenterological practice.” Scand. J. Gastroenterol. Suppl. 225: 13-8).

### LIST OF REFERENCES

1 radiation source
2 driver of the radiation source
3 light beam
3a first sub-beam
3b second sub-beam
4 cylindrical lens
5 first lens
6 second lens
7 filter
8 chopper
9 beam splitter
10 deviating mirror
11 third lens
12 first detector
13 sample chamber
14 gas inlet
15 gas outlet
16 gas flow meter
17 computer
18 mirror
19 fourth lens
20 second detector
21 first lock-in amplifier
22 second lock-in amplifier
D₁ signal of the first detector
D₂ signal of the second detector
S₁ first sub-signal of the signal of the first detector
S₂ first sub-signal of the signal of the second detector
S₃ second sub-signal of the signal of the first detector
S₄ second sub-signal of the signal of the second detector
S_{D} difference signal
ST₁ first voltage divider
ST₂ second voltage divider

1-31. (canceled)

32. A method for the spectroscopic analysis of a flowing gas, comprising steps:
continuously introducing a sample, which contains the gas to be analyzed, into a sample chamber and continuously releasing the sample from the sample chamber, the gas flow through the sample chamber being measured by any means for measuring the gas flow;
directing at least a part of radiation emitted by a radiation source through the sample chamber onto a detection apparatus by means of a system of optical elements for analysis of the gas, the sample being exposed to radiation with a wavelength in the range of from 4000 to 200 cm−1; and
detecting absorption of the radiation by the flowing gas to be analyzed by means of the detection apparatus, wherein the time resolution of the analysis is less than 0.3 sec.

33. The method as claimed in claim 32, wherein essentially only absorption of a single isotope of the gas is excited by the emitted radiation and recorded by the detection apparatus.

34. The method as claimed in claim 32, wherein the sample which contains the gas to be analyzed is respiratory air.

35. The method as claimed in claim 32, wherein the gas to be analyzed is ¹³CO₂.

36. The method as claimed in claim 32, wherein the sample is introduced into the sample chamber by means of a tube.

37. The method as claimed in claim 32, wherein the sample is released through an outlet means which only allows substance transport out of the sample chamber.

38. The method as claimed in claim 32, wherein the radiation source and/or at least one of the optical elements are configured for isotope-selective excitation of the absorption of the gas.

39. The method as claimed in claim 32, wherein the detection apparatus and/or at least one of the optical elements are configured for isotope-selective detection of the absorption of the gas.

40. The method as claimed in claim 32, wherein the radiation source is a quantum cascade laser, in particular with a repetition rate of 10 kHz.

41. The method as claimed in claim 32, wherein the detection apparatus is a photovoltaic MCT detector.

42. The method as claimed in claim 32, wherein the gas is analyzed in real-time.

43. The method as claimed in claim 32, wherein the sample is exposed to radiation in the wavelength range of from 2280 to 2230 cm⁻¹.

44. The method as claimed in claim 32, wherein the absolute concentration and total amount of an isotope of a gas is determined per time interval by the means for measuring the gas flow.

45. The method as claimed in claim 44, wherein the ¹³CO₂ concentration or total ¹³CO₂ concentration and ¹²CO₂ concentration or total amount of ¹²CO₂ are determined independently of one another.

46. The method as claimed in one of the preceding claims, usable for the determination of a biological parameter of an individual, wherein:
the gaseous sample to be analyzed originates from the individual; and
the biological parameter is:
the function of an organ of the individual;
the concentration of an enzyme in an organ and/or tissue of the individual; or
the concentration of at least one microbial species in an organ and/or tissue of the individual.
